(19) United States
(12) Patent Application Publication
(10) Pub. No.: US 2026/0240841 A1
(43) Pub. Date: Aug. 20, 2026

(54) COMBINATION OF HPK1 INHIBITOR AND AXL INHIBITOR IN CANCER THERAPY

(71) Applicant: BOARD OF REGENTS, THE UNIVERSITY OF TEXAS SYSTEM, Austin, TX (US)

(72) Inventors: Hua WANG, Houston, TX (US); Huamin WANG, Houston, TX (US)

(21) Appl. No.: 19/471,251

(22) PCT Filed: Mar. 29, 2024
(86) PCT No.: PCT/US2024/022298

§ 371 Date: Sep. 30, 2025

Related U.S. Application Data
(60) Provisional application No. 63/493,311, filed on Mar. 30, 2023.

(51) Int. Cl. A61K 31/502; A61K 31/506; A61K 31/519; A61K 39/00; A61K 39/395; A61P 35/00; C07K 16/28

(52) U.S. Cl. CPC A61K 31/502; A61K 31/506; A61K 31/519; A61K 39/39558; A61P 35/00; C07K 16/2878; A61K 2039/505

(57) ABSTRACT

Provided herein are methods of treating a subject that has cancer, using a combination therapy. The combination therapy includes an HPK1 inhibitor with an Axl inhibitor, optionally a Kras^{G12D}, and optionally an agonistic CD137 antibody. Also disclosed are diagnostic and prognostic methods for subjects having or suspected of having cancer and/or subjects undergoing treatment for cancer.

### CROSS REFERENCE TO RELATED APPLICATIONS

This application claims priority to and the benefit of U.S. Provisional Patent Application Ser. No. 63/493,311, filed Mar. 30, 2023. The entire contents of that application are incorporated herein by this reference as if fully set forth herein.

### STATEMENT AS TO RIGHTS TO INVENTIONS MADE UNDER FEDERALLY SPONSORED RESEARCH AND DEVELOPMENT

This invention was made with Government support under grant number R01CA196941 awarded by the National Institutes of Health. The Government has certain rights in the invention.

### BACKGROUND

The immune system plays an indispensable role in the maintenance of cellular homeostasis and the active inhibition of tumorigenesis. Unfortunately, cancers develop various mechanisms to bypass immune surveillance and evade the immune system. Recently, significant advances have been made in the field of cancer immunotherapy, which either restores or augments a patient's natural anti-tumor immune response, usually targeting specific biological molecules on cancer cells' surface. Immunotherapies have revolutionized cancer treatment. However, a majority of cancer patients have limited beneficial response to immunotherapies, for example, due to the presence of immune suppressive tumor microenvironments. Thus, developing new strategies for cancer treatment would be beneficial. For example, in some instances, methods that enhance the continuous recruitment of effector T cells to the tumor site to improve the systemic anti-tumor immunity would be beneficial for cancer patients, especially for patients with advanced cancer

### BRIEF SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect, provided herein are methods of treating a subject. The methods include administering a therapeutically effective amount of an Axl inhibitor and HPK1 inhibitor. In some embodiments, the HPK1 inhibitor is HPK1-IN-7 or GNE-1858. In some embodiments, the HPK1 inhibitor is selected from a group consisting of GNE-6893, BGB-15025, CFI-402411 and compound K (CompK). In some embodiments, the Axl inhibitor is bemcentinib. In some embodiments, the Axl inhibitor is selected from a group consisting of SLC-391, DS-1205c, gilteritinib, BPI-9016M, INCB081776, PF-07265807, Q702, TP-0903, and monoclonal antibodies YW327.6S2, D9, and E8. In some embodiments, the method further includes administering to the subject a therapeutically effective amount of a Kras^{G12D} inhibitor. In some embodiments, the methods include administering a therapeutically effective amount of a HPK1 inhibitor and a Kras^{G12D} inhibitor. In certain embodiments, the Kras^{G12D} inhibitor is MRTX1133. In some embodiments, the method may further include administering a therapeutically effective amount of a CD137 agonist antibody and/or a myeloid inhibitor.

In some embodiments, the subject has or is suspected of having cancer. In some embodiments, the cancer is selected from a group consisting of pancreatic, skin, lung, colon, prostate, breast, ovary, endometrial, liver, esophageal, stomach, and kidney.

In some embodiments, the therapeutically effective amount of the Axl inhibitor and the HPK1 inhibitor are administered at a first time point and are subsequently administered at least at a second time point. In some embodiments, the therapeutically effective amount of the Kras^{G12D} inhibitor is administered at a first time point and is subsequently administered at least at a second time point. In some embodiments, the therapeutically effective amount of the CD137 agonist antibody is administered at a first time point and is subsequently administered at least at a second time point. In some embodiments, the subject has previously failed to respond to an immune checkpoint inhibitor. In some embodiments, the subject has relapsed. In some embodiments, the method further includes at least a second anti-cancer therapy. In some embodiments, the second anti-cancer therapy is a chemotherapy, molecular targeted therapy, immunotherapy, gene therapy, surgery, hormonal therapy, epigenetic modulation, anti-angiogenic therapy, or cytokine therapy.

In another aspect, provided herein are methods of monitoring the response of a subject to a combination therapy, comprising measuring, in a sample from a subject, obtained at a first time point, an amount of expression of Axl, HPK1, or both to obtain a first expression level for Axl, HPK1, or both; administering to the subject a therapeutically effective amount of an Axl inhibitor and an HPK1 inhibitor; measuring in a sample from the subject, obtained at a subsequent time point, an amount of expression of Axl, HPK1, or both to obtain a second expression level of Axl, HPK1, or both; and comparing the first expression level to the second expression level to determine whether a change in expression level has occurred. In some embodiments, the HPK1 inhibitor is HPK1-IN-7 or GNE-1858. In some embodiments, the Axl inhibitor is bemcentinib. In some embodiments, the methods of monitoring further include measuring in a sample from the subject, obtained at the first time point, an amount of expression of Kras^{G12D} to obtain a first Kras^{G12D} expression level; optionally administering to the subject a therapeutically effective amount of a Kras^{G12D} inhibitor; measuring in a sample from the subject, obtained at the subsequent time point, an amount of expression of Kras^{G12D} to obtain a second expression Kras^{G12D} level; and comparing the first Kras^{G12D} expression level to the second Kras^{G12D} expression level to determine whether a change in Kras^{G12D} expression level has occurred. In some embodiments, the method of monitoring the response of a subject further comprises administering to the subject a therapeutically effective amount of a Kras^{G12D} inhibitor or CD137 agonist antibody before the subsequent time point. In some embodiments, the subject has or is suspected of having cancer. In some embodiments, the cancer is selected from a group consisting of pancreatic, lung, colon, prostate, breast, ovary, endometrial, liver, esophageal, stomach, skin, and kidney.

In some embodiments, the subject has previously failed to respond to an immune checkpoint inhibitor. In some embodiments, the subject has relapsed. In some embodiments, the method of monitoring further comprises administering to the subject at least a second anti-cancer therapy before the subsequent time point. In some embodiments, the second anti-cancer therapy is a chemotherapy, molecular targeted therapy, immunotherapy, gene therapy, surgery, hormonal therapy, epigenetic modulation, anti-angiogenic therapy, or cytokine therapy. In some embodiments, the Axl inhibitor and the HPK1 inhibitor are administered in at least two separate administrations between the first and subsequent time point. In some embodiments, the Kras^{G12D} inhibitor is administered in at least two separate administrations between the first and subsequent time point.

In another aspect, provided herein are methods of determining the likelihood that a subject will respond to an Axl-HPK1 inhibitor combination therapy, the method comprising (a) obtaining a sample from a subject and (b) detecting an expression level of HPK1 or Axl in the sample. In some embodiments, the method further comprises administering to the subject an HPK1 inhibitor and an Axl inhibitor if the expression level detected in step (b) is above a threshold level. In some embodiments, the expression level is a protein expression level. In some embodiments, the expression level is a nucleic acid expression level.

In another aspect, provided herein are methods of determining the likelihood that a subject will respond to an Axl-HPK1-Kras^{G12D} inhibitor combination therapy, the method comprising (a) obtaining a sample from a subject and (b) detecting an expression level of HPK1, Axl, or Kras^{G12D} in the sample. In some embodiments, the methods further include indicating that the subject is likely to respond to treatment if the subject's cancer is characterized as having high levels of HPK1, Axl, or Kras^{G12D} expression. In some embodiments, the method further comprises administering to the subject an HPK1 inhibitor, an Axl inhibitor, and/or a Kras^{G12D} inhibitor if the expression level detected in step (b) is above a threshold level. In some embodiments, the expression level is a protein expression level. In some embodiments, the expression level is a nucleic acid expression level.

In yet another aspect, provided herein are methods of modulating the microenvironment of a cell or plurality of cells in a subject, the method comprising administering, to the subject a therapeutically effective amount of a first kinase inhibitor and a second kinase inhibitor that modulate macrophages, CD8+ T cells, CD3+ T cells, CD4+ T cells, myeloid suppressor cells, or any combination thereof. In some embodiments, the subject has or is suspected of having a cancer selected from a group consisting of pancreatic, lung, colon, prostate, breast, ovary, endometrial, liver, esophageal, stomach, skin, and kidney. In some embodiments, the first kinase inhibitor is bemcentinib, and the second kinase inhibitor is HPK1-IN-7 or GNE-1858. In some embodiments, the first and second kinase inhibitors reduce the number or function of macrophage and myeloid suppressor cells. In some embodiments, the first kinase inhibitor and the second kinase inhibitor increase the number or function of CD8+ T cells, CD3+ T cells, or CD4+ T cells. In some embodiments, the method further comprises administering to the subject a therapeutically effective amount of a Kras^{G12D} inhibitor. In some embodiments, the method further comprises administering, to the subject, a therapeutically effective amount of a CD137 agonist antibody. In some embodiments, the subject has relapsed. In some embodiments, the method further comprises at least a second anti-cancer therapy. In some embodiments, the second anti-cancer therapy is a chemotherapy, molecular targeted therapy, immunotherapy, gene therapy, surgery, hormonal therapy, epigenetic modulation, anti-angiogenic therapy or cytokine therapy. In some embodiments, the combination of the Axl inhibitor and the HPK1 inhibitor is administered at least at two separate time points.

### BRIEF DESCRIPTION OF THE DRAWINGS

The present application includes the following figures. The figures are intended to illustrate certain embodiments and/or features of the compositions and methods, and to supplement any description of the compositions and methods. The figures do not limit the scope of the compositions and methods, unless the written description expressly indicates that such is the case.

FIGS. 1A-1B are images of Western blots of protein expression in the pancreas or spleen. FIG. 1A is a Western blot showing Axl and HSP90 expression in the pancreas of control L/IPF (LSL-Kras^{G12D}; Pdx-Cre) mice and L/IPF with HPK1 knockout (KO) mice (HPK1^{-/-}; LSL-Kras^{G12D}; Pdx-Cre). FIG. 1B is a Western blot of Axl and HSP90 expression in the spleen of HPK1 knockout mice (HPK1^{-/-}) and control mice (WT).

FIG. 2 is a graph showing the intra-tumoral cellular profile of hematopoietic cells collected from xenograft tumors from wild type (WT) and HPK1 knockout (KO) mice. The cells were stained with Zombie-UV and incubated with anti-CD45, anti-CD3ε, anti-B220, anti-CD11b, anti-F4/80, anti-Ly6C, anti-Ly6G, anti-CD4, anti-IA/IE, and anti-CD8α antibodies prior to analysis with multicolor flow cytometry. The y axis shows the percentage of CD45⁺ cells in the sample, and the x axis shows the different cell types present in the sample. In each pair of bars, the solid bar is from WT mice, and the open bar is from the KO mice.

FIGS. 3A-3B are exemplary IVIS bioluminescence images of orthotopic xenograft tumors. FIG. 3A shows bioluminescent images of orthotopic xenograft tumors taken 17 days after KPC-Luc cells implantation in untreated wild type (WT) mice (top panel) and WT mice treated with Axl inhibitor Bemcentinib (bottom panel). FIG. 3B shows bioluminescent images of orthotopic xenograft tumors taken 17 days after KPC-Luc cells implantation in HPK1 knockout (HPK1^{-/-}) mice (top panel) and HPK1^{-/-} mice treated with Axl inhibitor Bemcentinib (bottom panel).

FIGS. 4A-4B are bar graphs showing the intra-tumoral cellular profile of hematopoietic cells collected from wild type (WT, FIG. 4A) and HPK1 knockout (HPK1^{-/-}, FIG. 4B) mice according to certain embodiments of this disclosure. In FIGS. 4A-4B, hematopoietic cells were collected from WT mice control (left bar in each pair) and mice treated with Axl inhibitor (right bar in each pair) and subsequently stained with Zombie-UV and incubated with anti-CD45, anti-CD45, anti-CD3ε, anti-B220, anti-CD11b, anti-F4/80, anti-Ly6C, anti-Ly6G, anti-CD4, anti-IA/IE, and anti-CD8α antibodies prior to analysis with multicolor flow cytometry. The y axis shows the percentage of CD45+ cells in the sample, and the x axis shows the different cell types present in the sample.

FIGS. 5A-5B are exemplary heat maps depicting the expression levels of multiple immunosuppressive cytokines or markers for macrophages and myeloid suppressor cells. FIG. 5A is a heat map of expression levels in mouse pancreas, and FIG. 5B is a heat map of the expression levels in human pancreatic cancer samples.

FIGS. 6A-6B are bar graphs showing the effect of treatment with an Axl inhibitor, HPK1 inhibitor, or combination treatment on tumor growth. FIG. 6A is a graph showing the tumor volume measured from tumor samples collected from WT C57BL/6 mice after implantation with KPC-Luc cells (control) or after being treated with either HPK1 inhibitor, Axl inhibitor, or a combination of both inhibitors for 10 days and the tumor volumes were measured at 15 days after the completion of treatment. FIG. 6B is a graph of the tumor weight in grams measured from tumor samples collected from WT C57BL/6 mice after implantation with KPC-Luc cells (control) or after being treated with either HPK1 inhibitor, Axl inhibitor, or a combination of both inhibitors for 10 days. The tumors were harvested and weighed at 15 days after the completion of treatment.

FIG. 7 is a graph showing the effect of treatment with an Axl inhibitor (Axli) alone or in combination with a CD137 antibody and/or an HPK1 inhibitor (HPK1i) on tumor weight according to certain embodiments of this disclosure. Tumor samples were collected from WT C57BL/6 mice after implantation with KPC-Luc cells (control) or after being treated for 10 days with an Axl inhibitor or Axl inhibitor in combination with an HPK1 inhibitor (#1 or #2), in combination with CD137, or a combination of all three as indicated on the x axis. The tumors were harvested and weighed at 12 days after the completion of treatment.

FIGS. 8A-8B are images of Western blots showing protein expression in the spleen of C57BL/6 mice with orthotopic Kras^{G12D/+}; Trp53^{R172H/+}, Pdx-Cre; Luciferase (KPC-Luc) tumors. FIG. 8A is a Western blot showing HPK1 and Actin expression in the spleen of C57BL/6 mice with orthotopic KPC-Luc tumors with developed resistance to MRTX1133 (right three bands) and vehicle control (left three bands). FIG. 8B is a Western blot of Axl and HSP90 expression in the spleen of C57BL/6 mice with orthotopic KPC-Luc tumors sensitive to MRTX1133 (right three bands) and vehicle control (left two bands).

FIG. 9 provides exemplary IVIS bioluminescence images of orthotopic xenograft tumors in response to treatment with MRTX1133. Wild type (WT) mice (top panel) and HPK1. mice (bottom panel) with orthotopic xenograft tumors of KPC-Luc cells were treated with MRTX1133 (right group of 5 mice) or a vehicle control (left group of 5 mice).

FIG. 10 is a graph showing the effect of treatment with MRTX1133 in orthotopic KPC-Luc pancreatic cancer models. Wild type (WT) and HPK1^{-/-} mice were treated with 25 mg/kg (IP) of MRTX1133 twice a day for ten days and tumors were harvested and weighed at 7 days after completion of treatment

FIG. 11 is a graph showing the effect of treatment with MRTX1133 in subcutaneous KPC-Luc pancreatic cancer models. Wild type (WT) and HPK1^{-/-} mice were treated with 10 mg/kg (IP) of MRTX1133 twice a day for ten days (day 1 to 10). Tumor volume was measured continually from the start of treatment to 12 days post treatment.

FIGS. 12A-12B are bar graphs showing the effect of treatment with an Axl inhibitor, Bemcentinib, on subcutaneous B16F10 melanoma models. Wild type (WT) and HPK1^{-/-} mice after implantation with B16F10 melanoma cells (control) were treated with Axl inhibitor for ten days and tumors were harvested at 12 days after completion of treatment. FIG. 12A is a graph showing the tumor volume measured from tumor samples collected from WT C57BL/6 mice and HPK1−/− mice. FIG. 12B is a graph showing the tumor weight measured from tumor samples collected from WT C57BL/6 mice and HPK1−/− mice.

FIGS. 13A-13B are bar graphs showing the effect of treatment with an Axl inhibitor, HPK1 inhibitor, or combination treatment on tumor growth. WT C57BL/6 mice after implantation with B16F10 melanoma cells were treated with either vehicle (control), HPK1 inhibitor, Axl inhibitor, or a combination of both inhibitors for 10 days and the tumor volumes were measured at 12 days after the completion of treatment. FIG. 13A is a graph showing the tumor volume measured from tumor samples collected at 12 days after the completion of treatment. FIG. 13B is a graph showing the tumor weight at 12 days after the completion of treatment.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Introduction

The major challenge in the treatment of pancreatic ductal adenocarcinoma (PDAC) (as well as certain other cancers) is its resistance not only to conventional chemoradiation therapy, but also to the newly developed immunotherapy and targeted therapies due to the presence of dense desmoplasia and the immunosuppressive tumor microenvironment (TME). The disclosed methods of co-targeting HPK1 and Axl (alone or in combination with a CD137 agonist antibody and/or an immune checkpoint inhibitor (ICI) or gemcitabine or a Kras inhibitor (e.g., Rasi MRTX1133)) provide a strategy to improve the response of PDAC to ICIs and gemcitabine as well as providing novel insights into the immunosuppressive TME. In addition, because HPK1 inhibitors and Kras inhibitors are currently in clinical trials and the Axl inhibitor, bemcentinib, has been approved by FDA for Axl+ non-small cell lung cancer, the disclosed methods could be rapidly translated into new clinical trials, improving the clinical outcome and survival of PDAC patients. The disclosed treatment also is useful for diagnostic and prognostic purposes as well as for its application to other cancers.

### II. Terminology

Unless otherwise defined, all terms of art, notations, and other scientific or medical terms or terminology used herein are intended to have the meanings commonly understood by those of ordinary skill in the art. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e., at least one) of the grammatical object of the article. By way of example, “an element” means at least one element and can include more than one element.

The use herein of the terms “including.” “comprising.” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of those certain elements.” As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, e.g., In re Herz, 537 F. 2d 549, 551-52 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20%; preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X” are intended to teach and provide written support for a claim limitation of, for example, “0.98X.” Numerical quantities given herein are approximate unless stated otherwise, meaning that the term “about” or “approximately” can be inferred when not expressly stated. When “about” is applied to the beginning of a numerical range, it applies to both ends of the range.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2, and/or 3” is equivalent to “‘1’ or ‘2’ or ‘3’ or ‘1 and 2’ or ‘1 and 3’ or ‘2 and 3’ or ‘1, 2, and 3.’” Whenever a range is given in the specification, for example, a temperature range, a time range, or a composition range, all intermediate ranges and subranges, as well as all individual values included in the ranges given are intended to be included in the disclosure.

All patents, patent applications, publications, and descriptions mentioned herein are incorporated by reference in their entirety for all purposes. None is admitted to be prior art. Where a conflict exists between the instant application and a reference provided herein, the instant application shall dominate.

### III. PDAC, Axl, HPK1, KRAS, and CD137

PDAC is one of the most lethal tumors among all malignancies and ranks as the third leading cause of cancer-related deaths in the United States. Pancreatic cancer is resistant to conventional chemotherapy and radiation therapies. The 5-year survival rate for patients with pancreatic cancer is ~8.5 % (Are et al., Surg. Oncol. 115:637-641, (2017)). Currently, the mainstay of treatment for metastatic PDAC is chemotherapy with gemcitabine-or fluorouracil-based regimens; however, chemotherapy benefits are often modest and transient (National Cancer Institute SRP (2021); Rahib et al., Est. Proj. of U.S. Cancer, (2021)). While immune checkpoint inhibitors (ICIs) have transformed the treatment and improved the survival for many other cancers, PDAC has limited response to ICIs, including anti-PD1/PD-L1, anti-CTLA4 and combined anti-PD1+anti-CTLA4 (Bear et al., Cancer Cell, 38:788-802 (2020); Timmer et al., Cancers, 13:1-42 (2021). This is likely due to the presence of an immunosuppressive tumor microenvironment (TME) in PDAC, which helps the tumor cells evade from the host antitumor immunity and promotes tumor progression (Blando et al., Proc. Natl. Acad. Sci. U.S.A., 116:1692-1697 (2019); Chen et al., Cancer Cell, 39:548-565 (2021); Ma et al., Gastroenterology, 159:306-319 (2020)).

Oncogenic receptor tyrosine kinase Axl plays a major role in cancer cell survival, proliferation, migration, invasion, metastasis, and immunosuppressive tumor microenvironment (Ludwig et al., Cancer Res., 78:246-255 (2018); Skinner et al., Clin. Cancer Res., 23:2713-2722 (2017); Zhang et al., Clin. Cancer Res., 24:4771-4784 (2018)). Axl and its ligand Gas 6 are overexpressed in pancreatic cancer and carcinomas of other organs, including lung, colon, prostate, breast, ovary, esophagus, stomach, and kidney (Hutterer et al., Clin Cancer Res., 14: 130-138 (2008); O'Brian et al., Mol. Cell Biol,, 11:5016-5031 (1991); Vajkoczy et al., Proc. Natl. Acad. Sci. U.S.A., 103:5799-5804 (2006); Chung et al., DNA Cell Biol., 22:533-540 (2003); Craven et al., Int. J. cancer, 60:791-797 (1995); Meric et al., Clin. Cancer Res., 8:361-367 (2002); Nakano et al., Clin. Exp. Metastasis, 20:665-674 (2003); Nemoto et al., Pathology, 65:195-203 (1997); Shieh et al., Neoplasia, 7:1058-1064 (2005); Van Ginkel et al., Cancer Res., 64:128-134 (2004); Wu et al., Anticancer Res., 22:1071-1078 (2002); Zhang et al., Cancer Res., 68:1905-1915 (2008); Song et al., Cancer, 117:734-743 (2011); Koorstra et al., J. Clin. Invest., 127:183-198 (2017)). Recently, it was revealed that Axl is overexpressed in 70% of human pancreatic cancer samples and that overexpression of Axl correlated significantly with a higher rate of distant metastasis, shorter recurrence-free survival, and overall survival in patients with pancreatic cancer who underwent upfront pancreatectomy with curative intent (Song et al., Cancer, 117:734-743 (2011)). Axl silencing sensitizes pancreatic cancer cells to γ-irradiation and reduces their anchorage-independent growth, migration, as well as invasion potential, which has been attributed to the down-regulation of AKT signaling as well as transcription factors involved in epithelial-to-mesenchymal transition (EMT) such as slug, snail, and twist, etc. (Song et al., Cancer, 117:734-743 (2011); Koorstra et al., J. Clin. Invest., 127:183-198 (2017)). Subsequent studies targeting the Gas6-Axl-signaling pathways using either a small molecular inhibitor (BGB324), a high-affinity Axl decoy receptor, or neutralizing monoclonal antibodies (mAbs) against Axl or Gas6 demonstrate that autocrine Gas6-Axl signaling is an important driver for therapeutic resistance, disease progression, and metastasis in pancreatic cancer and other human malignancies (Ludwig et al., Cancer Res., 78:246-255 (2018); Kariolis et al., J. Clin. Invest., 127:183-198 (2017); Kirane et al., Cancer Res., 75:3699-3705 (2015); Leconet et al., Clin. Cancer Res., 23:2806-2816 (2017); Leconet et al., Oncogene, 33:5405-5414 (2014); Moody et al., Int. J. Cancer, 139:1340-1349 (2016); Antony et al., EMBO Rep, 19: (2016); Goyette et al., Cell Rep., 23:1476-1490 (2018), Paolino et al., Nature, 507:508-512 (2014)). Ludwig et al. demonstrated that the selective Axl kinase inhibitor, BGB324, not only inhibits the aggressiveness of pancreatic cancer and sensitizes pancreatic cancer cells to gemcitabine, but also induces an immune stimulatory microenvironment. Neutralizing mAbs against Axl or Gas6 inhibits AKT signaling in pancreatic cancer and in vivo tumor growth in xenograft tumor models (Ludwig et al., Cancer Res., 78:246-255 (2018); Leconet et al., Oncogene, 33:5405-5414 (2014)). The results from these preclinical studies provide strong rationale for targeting the Gas6-Axl autocrine pathway to improve the treatment efficacies and clinical outcomes in patients with pancreatic cancer and cancers of other organs.

Previous studies by Leconet et al. (Clin. Cancer Res., 23:2806-2816 (2017); Oncogene, 33:5405-5414 (2014)) showed that anti-Axl mAbs induced internalization and down-regulation of Axl in both pancreatic cancer and triple-negative breast cancer cells. Their results suggest that endocytosis may be one of the major mechanisms regulating Axl expression in cancer cells. Consistent with this notion, Valverde (Biochem. Biophys. Res. Com. 333:180-185 (2005)) reported that binding of Gas6 to Axl induces the phosphorylation, ubiquitination, and down-regulation of Axl in human lens epithelial cells through endocytosis/lysosomal degradation, but not through proteasomal degradation. However, the molecular mechanisms regulating the expression of Axl in pancreatic cancer or other human malignancies are unclear.

Hematopoietic progenitor kinase 1 (HPK1), also named MAP4K1, is a mammalian Ste20-related serine/threonine kinase, which has been shown to regulate NF-KB and c-Jun N-terminal kinase pathways in hematopoietic cells (Arnold et al., J. Biol. Chem., 276:14675-14684 (2001); Chen et al., Oncogene, 18:7370-7377 (1999)). HPK1 kinase activity plays an important role in regulating T cell function mainly through the activation of NF-KB and c-Jun N-terminal kinase and inhibition of the MEK1/2-mediated Erk activation (Shui et al., Nature Immun., 8:84-91 (2007); Si et al., Cancer Cell, 38:551-566 (2020)). Inhibition of HPK1 kinase function by knocking in the kinase-dead mutant of HPK1, M46, in which the ATP binding lysine-46 residue was mutated to methionine, increases the T cell receptor signaling, cytokine secretion, and CD8+ T cell function, and inhibits tumor growth in M46 transgenic mice. Similarly, knockout HPK1 or inhibition of HPK1 kinase activity using small molecular inhibitors has also been shown to enhance the T cell antitumor functions and to inhibit tumor growth in syngeneic allograft mouse models (Si et al., Cancer Cell, 38:551-566 (2020); Wang et al., PLoS One, 15:e0243145 (2020); You et al., J Immunother. Cancer, 9: (2021). While HPK1 kinase inhibitors can block HPK1 kinase activity with the half maximal inhibitory concentration (IC50) values in the low nM range, there is no published report on the efficacy of HPK1 inhibitor to enhance antitumor immunity in clinical trials.

While it is generally believed that HPK1 is expressed at high levels only in hematopoietic cells, its expression is not exclusively restricted to hematopoietic cell compartments. HPK1 is also expressed and associated with the oncogenic receptor tyrosine kinase, Axl, in pancreatic precursor lesions and pancreatic cancer cells (Song et al., J. Biol. Chem. 295(8): 2348-2358 (2020). These data showed that HPK1 may negatively regulate Axl in lymphocytes, dendritic cells, and macrophages. The GAS6/Axl plays a major role in promoting an immune suppressive tumor microenvironment. Targeting HPK1 using an HPK1 inhibitor may up-regulate Axl, which in turn blocks the immunogenic tumor microenvironment and reduces the efficacy of combined therapy of HPK1 inhibitors with or without immune checkpoint inhibitors (ICIs).

Clinical trials to target HPK1 kinase activity in immune cells for cancer immunotherapy are currently ongoing. Treadwell Therapeutics advanced an HPK1 inhibitor, CFI-402,411, to the phase 1/2 clinic trial in 2020. The structure of CFI-402,411 was not disclosed. The company has shared pre-clinical data at conferences to illustrate anti-tumor activity in several murine models as a potential monotherapy and in combination with ICIs for both solid and hematological cancers. In addition, tumor re-challenge in animals with complete response shows no re-growth, consistent with immunologic memory. In this phase 1/2 study, CFI-402,411 is administered as a single agent or in combination with pembrolizumab (anti-PD1 antibody). The second company, Beigene, started their HPK1 inhibitor, BGB-15,025, into Phase 1 in January 2021.

Kirsten rat sarcoma viral oncogene homologue (KRAS) is a GTPase known to be involved in the regulation of cell division. However, in cancer tissues, KRAS mutations occur more readily and the KRAS protein may remain in an active state even in the absence of growth signals. One such mutant variant (KrasG12D or Kras^{G12D}) is present in more than 90% of pancreatic cancers and is present in multiple other cancer types. In early pancreatic intraepithelial neoplasias, increased expression of Kras^{G12D} mutations is linked to increased invasion and metastasis to PDAC. Interestingly, it has been demonstrated that wild-type HPK1 inhibits Ras activation through the upregulation of RasGAP activity and inhibits Kras^{G12D}-driven development in pancreatic intraepithelial neoplasias (Wang et al., J. Clin. Invest. 133(12): e163873 (2023)). Thus, inhibiting HPK1 increases the expression of KrasG12D, potentially leading to growth of PDAC in patients. Clinical testing of a Kras^{G12D} inhibitor, MRTX1133, is currently ongoing in solid tumor models. Experimental testing of MRTX1133 has demonstrated the ability of the Kras^{G12D} inhibitor to shrink tumors or halt the growth in mouse PDAC models.

CD137 is also known as 4-1BB and is co-stimulatory immune receptor that is a member of the tumor necrosis factor receptor (TNFR) superfamily. CD137 is expressed on activated T cells, NK cells, dendritic cells, eosinophils, mast cells, endothelial cells, and some tumor cells. CD137 monoclonal antibodies have demonstrated improved antitumor T-cell responses in some studies. See, e.g., Sharma et al., Clin. Immunology (5^{th} ed.), Ch. 77 Immunotherapy of Cancer (2019).

The present application inventors further explored the mechanisms of the tumor suppressor functions of HPK1. Axl was identified as one of the major HPK1-interacting proteins in PDAC cells using antibody array-based screening, and the role of HPK1 in regulating Axl signaling was studied. A novel mechanism by which HPK1 down-regulates oncogenic Axl through the endocytic pathway was revealed, but also provides the new link between HPK1 and the oncogenic Gas6-Axl pathway in pancreatic cancer. HPK1 interacted with the SH3 domain and phosphorylated Ras GTPase-activating protein (RasGAP), upregulated RasGAP activity and inhibits Ras activity (Wang et al., J. Clin. Invest. 133(12): e163873(2023 )). Thus, a combination therapy targeting HPK1 and Axl (alone or in combination with a CD137 agonist antibody and/or a Kras^{G12D} inhibitor) could regulate the tumor microenvironment to convert an immune “cold” tumor (i.e., a tumor that is not likely to trigger a strong immune response) to an immune “hot” tumor (i.e., a tumor that is likely to trigger a strong immune response). The combination therapy of an HPK1 inhibitor and Axl inhibitor (alone or in combination with a CD137 agonist antibody and/or a Kras^{G12D} inhibitor (e.g., Rasi MRTX1133)) would have durable and markedly improved antitumor efficacy and provide urgently needed therapeutic options for pancreatic cancer and other cancers. In addition, a combination therapy targeting HPK1 and Kras^{G12D} may provide needed therapeutic options for pancreatic cancer and other cancers.

### IV. Methods of Use

Provided herein are methods to treat or inhibit a disease or disorder associated with Kras mutation or elevated levels of Axl or HPK1. In some embodiments, the disease or disorder is a cancer, such as pancreatic, lung, colon, prostate, breast, ovary, endometrial, liver, esophageal, stomach, skin, or kidney cancer. The methods include reducing or inhibiting the functioning of Axl and HPK1 in the subject. Such reduction or inhibition may be accomplished by any suitable treatment that specifically binds to and/or otherwise inhibits the function of Axl or HPK1. Preferably, such treatment includes administering to a subject a therapeutically effective amount of an Axl inhibitor and an HPK1 inhibitor as described herein. Other suitable combination treatments may include an Axl inhibitor, an HPK1 inhibitor, and a CD137 agonist antibody or immune check point inhibitor. In some embodiments, other suitable combination treatments may include an HPK1 inhibitor and a Kras^{G12D} inhibitor. In some embodiments, the combination therapy may include an HPK1 inhibitor, an Axl inhibitor, a Kras^{G12D} inhibitor, and a CD137 agonist antibody or immune check point inhibitor. In some embodiments, the combination therapy may include a myeloid cell inhibitor (CXCR2, CXCR4 inhibitors, and CD11b inhibitors), an HPK1 inhibitor, an Axl inhibitor, a Kras^{G12D} inhibitor, a CD137 agonist antibody or immune check point inhibitor, or any combination thereof.

Prognostic and diagnostic methods for cancer are also provided, based on detection and/or quantitation of Axl or HPK1 expression. Also provided are methods of modulating the tumor microenvironment of a cell or plurality of cells, comprising administering to the subject an effective amount of a first kinase inhibitor and a second kinase inhibitor.

### A. Methods of Treatment

Provided herein are methods to treat or inhibit a disease or disorder in a subject in need thereof using an Axl inhibitor and HPK1 inhibitor, and optionally a CD137 agonist antibody, as described in this disclosure. In some embodiments, the methods to treat or inhibit a disease or disorder in a subject in need thereof may include an Axl inhibitor, an HPK1 inhibitor, a Kras^{G12D} inhibitor, and optionally a CD137 agonist antibody. In other embodiments, the method may include using an HPK1 inhibitor and a Kras^{G12D} inhibitor, and optionally a CD137 agonist antibody. In some embodiments, the disease or disorder is a cancer. As used herein the terms “cancer” and “tumor” are used to indicate the presence of abnormal cells that proliferate uncontrollably and can invade nearby tissues or to describe malignant cells and tissue. The term “cancer” is also used to refer to the disease associated with the presence of malignant tumor cells in an individual, and the term “tumor” is used to refer to a plurality of cancer cells that are physically associated with each other. Cancer cells are malignant cells that give rise to cancer, and tumor cells are malignant cells that can form a tumor and thereby give rise to cancer. Cancer may describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, stomach, colon, rectum, anus, gum, head, kidney, liver, lung, nasopharynx, neck, ovary, pancreas, prostate, skin, stomach, testis, tongue, or uterus. In certain embodiments, the cancer is pancreatic, skin, lung, colon, prostate, breast, ovary, esophageal, stomach, or kidney cancer.

As used throughout, “subject” can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably, and the term patient or subject includes human and veterinary subjects. The methods of treatment described herein are useful for treating cancer in humans, including, without limitation, pediatric and geriatric populations, and in animals, e.g., veterinary applications. In one embodiment, the subject is a human.

As used herein, an “effective amount” means the amount of an agent that is effective for producing a desired effect in a subject. The actual dose that comprises the effective amount may depend upon the route of administration, the size and health of the subject, the disorder being treated (e.g., cancer), and the like.

The methods include reducing or inhibiting the functioning of Axl and HPK1 in the subject. Such reduction or inhibition may be accomplished by any suitable treatment that specifically binds to and/or otherwise inhibits the function of Axl or HPK1. Preferably, such treatment includes a combination therapy of an Axl inhibitor and an HPK1 inhibitor. Another suitable combination treatment may include an Axl inhibitor, an HPK1 inhibitor, and a Kras^{G12D} inhibitor. Other suitable combination treatments may include an Axl inhibitor, an HPK1 inhibitor, and a CD137 agonist antibody and/or a Kras^{G12D} inhibitor. In some embodiments, the inhibitor may be a small molecule inhibitor. In some embodiments, the inhibitor is a monoclonal antibody.

In one embodiment, the combination therapy of an Axl inhibitor, HPK1 inhibitor, optionally a Kras^{G12D} inhibitor, and optionally a CD137 agonist antibody, can directly inhibit growth and induce cell death of cancer cells. In some instances, the Axl inhibitor and HPK1 inhibitor and optionally the Kras^{G12D} inhibitor may directly bind to their respective target, inhibiting their activity. In some instances, the inhibitor may bind to any region of the protein to inhibit activity. For example, the inhibitor may bind to the intracellular catalytic kinase domain. In some instances, the Axl inhibitor in combination with an HPK1 inhibitor, optionally a Kras^{G12D} inhibitor, and optionally a CD137 agonist antibody, can sensitize cancer cells to other cancer therapies (e.g., chemotherapy). In some instances, treating a subject according to the methods described herein inhibits at least one of formation of a tumor, the proliferation of tumor cells, the growth of tumor cells, survival of tumor cells in circulation, or metastasis of tumor cells in the individual. In another embodiment, treating a subject according to the methods described herein may result in tumor growth stasis, reduction of tumor size and, in some instances, elimination of one or more tumors in the subject.

In some embodiments, the Axl inhibitor can be any one of SLC-391, DS-1205c, gilteritinib, BPI-9016M, INCB081776, PF-07265807, Q702, TP-0903, bemcentinib, PROTAC Axl Degrader 1, PROTAC Axl Degrader 2, enapotamab, Axl-IN-3, Axl-IN-4, Axl-IN-5, Axl-IN-6, Axl-IN-7, Axl-IN-8, Axl-IN-9, Axl-IN-10, Axl-IN-11, Axl-IN-12, Axl-IN-13, Axl-IN-14, and monoclonal antibodies YW327.6S2, D9, and E8. In certain embodiments, the Axl inhibitor is bemcentinib. In some embodiments, the HPK1 inhibitor can be any one of GNE-6893, BGB-17 15025, CFI-402411, compound K (CompK), HPK1-IN-7, HPK1-IN-21, HPK1-IN-32, HPK1-IN-24, HPK1-IN-8, HPK1-IN-26, HPK1-IN-25, HPK1-IN-28, HPK1-IN-29, HPK1-IN-31, HPK1-IN-13, HPK1-IN-12, HPK1-IN-14, HPK1-IN-9, HPK1-IN-11, HPK1-IN-10, HPK1-IN-4, HPK1-IN-20, HPK1-IN-19, HPK1-IN-27, HPK1-IN-30, HPK1-IN-3, HPK1-IN-15, HPK1-IN-16, HPK1-IN-17, HPK1-IN-18, GNE-1858, or ZYF0033. In certain embodiments, the HPK1 inhibitor is HPK1-IN-7 or GNE-1858. In some embodiments, the CD137 agonist antibodies can be any one of Urelumab (BMS-663513), AGEN2373, Utomilumab (PF-05082566), or Tecaginlimab (BNT-312, GEN1042). In some embodiments, the Kras^{G12D} inhibitor is MRTX1133.

Administration of the Axl inhibitor, HPK1 inhibitor, Kras^{G12D} inhibitor, and/or CD137 agonist antibody described herein can be carried out using therapeutically effective amounts of the molecules as described herein for periods of time effective to treat a disorder. The effective amount of the active ingredient (e.g., small molecule inhibitor or antibody) or pharmaceutically acceptable salts thereof as described herein may be determined by one of ordinary skill in the art and includes exemplary dosage amounts for a mammal of from about 0.0001 to about 200 mg/kg of body weight of active ingredient per day, which may be administered in a single dose or in the form of individual divided doses, such as from 1 to 4 times per day. Alternatively, the dosage amount can be from about 0.01 to about 150 mg/kg of body weight of active ingredient per day, about 0.1 to 100 mg/kg of body weight of active ingredient per day, about 0.5 to about 75 mg/kg of body weight of active ingredient per day, about 0.5 to about 50 mg/kg of body weight of active ingredient per day, about 0.01 to about 50 mg/kg of body weight of active ingredient per day, about 0.05 to about 25 mg/kg of body weight of active ingredient per day, about 0.1 to about 25 mg/kg of body weight of active ingredient per day, about 0.5 to about 25 mg/kg of body weight of active ingredient per day, about 1 to about 20 mg/kg of body weight of active ingredient per day, about 1 to about 10 mg/kg of body weight of active ingredient active ingredient per day, about 20 mg/kg of body weight of active ingredient per day, about 10 mg/kg of body weight of active ingredient per day, about 5 mg/kg of body weight of active ingredient per day, about 2.5 mg/kg of body weight of active ingredient per day, about 1.0 mg/kg of body weight of active ingredient per day, or about 0.5 mg/kg of body weight of active ingredient per day, or any range derivable therein. Optionally, the dosage amounts are from about 0.01 mg/kg to about 10 mg/kg of body weight of active ingredient per day. Optionally, the dosage amount is from about 0.01 mg/kg to about 5 mg/kg. Optionally, the dosage amount is from about 0.01 mg/kg to about 2.5 mg/kg.

Those of ordinary skill in the art will understand that the specific dose level and frequency of dosage for any particular subject may be varied and will depend upon a variety of factors, including the activity of the specific active ingredient employed, the metabolic stability and length of action of that active ingredient, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

Depending on the intended mode of administration, and as described further below, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions include a therapeutically effective amount of the active ingredients described herein or derivatives thereof in combination with a pharmaceutically acceptable carrier and, in addition, may include other medicinal agents, pharmaceutical agents, carriers, or diluents. The phrase “pharmaceutically acceptable” refers to a material that is not biologically or otherwise undesirable, which can be administered to an individual along with the selected compound without causing unacceptable biological effects or interacting in a deleterious manner with the other components of the pharmaceutical composition in which it is contained.

In some instances, the provided methods may include administering a combination therapy of an Axl inhibitor, an HPK1 inhibitor, optionally a Kras^{G12D} inhibitor, and optionally a CD137 agonist antibody, and a second form of cancer therapy to the subject. The second form of cancer therapy may include a cytotoxic agent, a chemotherapeutic agent, a radiotherapeutic agent, a phototherapeutic agent, an immunosuppressive agent (including immune checkpoint inhibitors), or radiation therapy. In some embodiments, the second form of cancer therapy is an antibody (e.g., a monoclonal antibody).

The combination therapies may enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with the combination therapy and a second therapy. A tissue, tumor, or cell can be contacted with one or more compositions or pharmacological formulation(s) comprising one or more of the agents (i.e., antibody or antibody fragment, an anti-cancer agent, or small molecule inhibitors), or by contacting the tissue, tumor, and/or cell with two or more distinct compositions or formulations, wherein one composition provides 1) a combination therapy of a first and second kinase inhibitor, 2) an anti-cancer agent, or 3) both a first and second kinase inhibitor and an anti-cancer agent. Also, it is contemplated that such a combination therapy can be used in conjunction with chemotherapy, external beam radiotherapy, surgical therapy, immunotherapy, or radioimmunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

The combination therapy of an Axl inhibitor with an HPK1 inhibitor, optionally a Kras^{G12D} inhibitor, and optionally a CD137 agonist antibody may be administered before, during, after, or in various combinations relative to an anti-cancer treatment. The administrations may be intervals ranging from concurrently to minutes to days to weeks. In embodiments where the combination of an Axl inhibitor with an HPK1 inhibitor, optionally a Kras^{G12D} inhibitor, and optionally a CD137 agonist antibody is provided to a subject separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 hours of each other and, more particularly, within about 6-12 hours of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some instances, the treatment methods provided herein may further comprise administering an immunosuppressive agent such as an immune checkpoint inhibitor as part of the method. These treatments work by “taking the brakes off” the immune system (are immunosuppressive), allowing it to mount a stronger and more effective attack against cancer. Several different types of checkpoint inhibitors, targeting different checkpoints or “brakes” on immune cells, are currently in use. Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCRS, glucocorticoid-induced tumor necrosis factor receptor-related protein (GITR), HLA-DRB 1, ICOS (also known as CD278), HLA-DQA1, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-LI, also known as CD274), PDCDILG2, PSMB 10, ST A T, T cell immunoreceptor with lg and ITIM domains (TI GIT), T-cell immunoglobulin domain and mucin domain 3 (TIM-3), and V-domain 1g suppressor of T cell activation (VISTA, also known as C10orf54). In particular, the immune checkpoint inhibitors target the PD-1 axis and/or CTLA-4. Exemplary immunosuppressive agents are PD-1 inhibitors (such as nivolumab and pembrolizumab), PD-L1 inhibitors (such as atezolizumab, durvalumab, and avelumab), and CTLA-4 inhibitors (such as ipilimumab). In one example, the second form of cancer therapy comprises a PD-L1 inhibitor, a PD-1 inhibitor, or a CTLA4 inhibitor. In some instances, combinations of such inhibitors can be administered. In some instances, the PD-L1 inhibitor, the PD-1 inhibitor, and/or the CTLA4 inhibitor may be an inhibitory antibody that binds specifically to PD-L1, PD-1, or CTLA4, respectively.

In some instances, the treatment methods provided herein may further comprise administering radiation therapy to the subject. Radiation therapy uses high-energy radiation to shrink tumors and kill cancer cells. X-rays, gamma rays, and charged particles are types of radiation used for cancer treatment. The radiation may be delivered by a machine outside the body (external-beam radiation therapy), or it may come from radioactive material placed in the body near cancer cells (internal radiation therapy, also called brachytherapy). Systemic radiation therapy uses radioactive substances, such as radioactive iodine, that travel in the blood to kill cancer cells.

“Treat,” “treatment,” and the like are used herein to generally mean obtaining a desired pharmacologic and/or physiologic effect. “Treating” or “treatment” may refer to any indicia of success in the treatment or amelioration of cancer. “Treating” or “treatment” includes the administration of an agent to impede growth of a cancer, to do one or more of the following: cause a cancer to shrink by weight or volume, extend the expected survival time of the subject, or extend the expected time to progression of the tumor, or the like. The effect of treatment can be compared to an individual or pool of individuals not receiving the treatment, or to the same patient prior to treatment or at a different time during treatment.

The term “administer,” as used herein, refers to a method of delivering agents, compounds, or compositions to the desired site of biological action. The pharmaceutical compositions (e.g., as described above) are prepared for administration in a number of ways, including but not limited to injection, ingestion, transfusion, implantation, or transplantation, depending on whether local or systemic treatment is desired, and on the area to be treated. The preparation of such pharmaceutically acceptable compositions is within the ability of one skilled in the art. The compositions are administered via any of several routes of administration, including topical, oral, parenteral, intravenous, intra-articular, intraperitoneal, intramuscular, subcutaneous, intracavity, intralesional, transdermal, intradermal, intrahepatical, intrathecal, intracranial, rectal, transmucosal, intestinal, ocular, intra-ocular, otic, nasal, inhalation, or intrabronchial delivery, or any other method known in the art. In some embodiments, an Axl inhibitor, an HPK1 inhibitor, optionally a Kras^{G12D} inhibitor, and optionally a CD137 agonist antibody are each independently administered orally, intravenously, or intraperitoneally. In some embodiments, an HPK1 inhibitor and a Kras^{G12D} inhibitor are each independently administered orally, intravenously, or intraperitoneally.

In another aspect, methods for modulating a microenvironment of a cell or plurality of cells in a subject are provided. In some embodiments, the method includes administering to the subject an effective amount of a first kinase inhibitor and a second kinase inhibitor that modulate macrophages, CD8+ T cells, CD3+ T cells, CD4+ T cells, myeloid suppressor cells, or any combination thereof. In some embodiments, the first kinase inhibitor is an Axl inhibitor, and the second kinase inhibitor is an HPK1 inhibitor. In some embodiments, the Axl inhibitor is bemcentinib. In some embodiments, the HPK1 inhibitor is HPK1-IN-7 or GNE-1858. As used herein the term “modulate” is used to indicate a change in the microenvironment. For example, in some embodiments, modulating the microenvironment includes increasing the number or function of certain cells. The term “modulate” is also used herein to refer to the ability of the inhibitors to exert a modifying or controlling influence on the cells treated with the combination therapy. In some embodiments, the first and second kinase inhibitors reduce the number or function of macrophage and myeloid suppressor cells. In some embodiments, the first kinase inhibitor and the second kinase inhibitor increase the number or function of CD8+ T cells, CD3+ T cells, or CD4+ T cells. In some embodiments, the method of modulating the microenvironment further includes administering a therapeutically effective amount of a Kras^{G12D} inhibitor and/or a CD137 agonist antibody.

In some embodiments, the modulation of the tumor microenvironment may “re-sensitize” the cell or plurality of cells to an existing cancer therapy such as a chemotherapy, molecular targeted therapy, immunotherapy, gene therapy, surgery, hormonal therapy, epigenetic modulation, anti-angiogenic therapy or cytokine therapy.

The term “re-sensitize” can include modulate or modulation in terms of the tumor microenvironment. In other embodiments, re-sensitize may refer to converting a “cold” tumor microenvironment to a “hot” tumor microenvironment. A cold tumor microenvironment is used to describe a microenvironment of a cell or plurality of cells that are not likely to trigger a strong immune response. Cold TME's are typically surrounded by cells that are able to suppress the immune response and keep T-cells from attacking the tumor cells. Cold TME's typically do not respond to immunotherapy or have weak response. To modulate the “cold” TME to a “hot” TME, the T-cells may be able to attack the tumor cells and immune suppression would thus be weakened. The cells in a “hot” TME are likely to respond to other immunotherapies. In some embodiments, methods described herein for treatment with an HPK1 inhibitor, an Axl inhibitor, and optionally a CD137 agonist antibody may convert a “cold” TME to a “hot” TME, thus inhibiting the growth of cancer cells.

### B. Diagnostic Methods

In another aspect, methods are provided for assessing eligibility for inclusion in or exclusion from a clinical trial and/or assessing the likelihood of response of a subject to a combination therapy including an Axl inhibitor, an HPK1 inhibitor, optionally a Kras^{G12D} inhibitor, and optionally a CD137 agonist antibody, alone or in any combination. In some embodiments, the method includes (a) obtaining a blood sample, a tumor sample, serum sample, CSF sample, or accessible fluid sample from a subject; (b) detecting an expression level of HPK1 and/or Axl in the sample; and (c) determining that the subject is likely to respond to treatment if the subject's cancer is characterized as having a high level of HPK1 and/or Axl expression (i.e., at or above a predetermined threshold level of expression). The methods may include exclusion of a subject for a clinical trial of the combination therapy if the subject's cancer is characterized as having a low level of Axl or HPK1 expression (i.e., below a predetermined threshold). In some embodiments, the threshold level is a median amount of HPK1 or Axl determined in a reference population of subjects having the same kind of cancer as the subject. In another embodiment, the threshold level is an optimal amount of Axl or HPK1 determined in a reference population of patients having the same kind of cancer as the subject. Conversely, if the subject's cancer is characterized as having a low level of Axl or HPK1 expression, the subject is less likely to respond to a combination therapy of an Axl inhibitor and HPK1 inhibitor optionally with a Kras^{G12D} inhibitor and/or optionally with a CD137 agonist antibody. In some instances, the amount of Axl or HPK1 in the tumor sample is measured using an antibody that specifically binds to the respective target. In some embodiments, the methods also determine the expression level of Kras^{G12D}.

“Optimal cutoff” or “optimal amount” as used herein, refers to the value of a predetermined measure on subjects exhibiting certain attributes that allow the best discrimination between two categories of an attribute. For example, finding a value for an optimal cutoff that allows one to best discriminate between two categories (subgroups) of patients for determining at least one of overall survival, time to disease progression, progression-free survival, and likelihood to respond to treatment (e.g., based on clinical assessment using the RECIST criteria, e.g., Eisenhauer, E.A., et al., Eur. J. Cancer 45:228-247 (2009) or the like as recognized in the medical field). Optimal cutoffs are used to separate the subjects with values lower than or higher than the optimal cutoff to optimize the prediction model, for example, without limitation, to maximize the specificity of the model, maximize the sensitivity of the model, maximize the difference in outcome, or minimize the p-value from hazard ratio or a difference in response.

Methods of detecting the expression level can be any method of detecting protein levels known to one skilled in the art. The method could also be any known method of detecting nucleic acids in a sample to one skilled in the art. Non-limiting examples can include, ELISA assays, Western blot, any applicable protein concentration assay, PCR, and DNA or RNA quantification.

In another aspect, provided are methods of monitoring the response of a subject with cancer to a combination therapy. In some embodiments, the methods include: (a) measuring in a sample from a subject, obtained at a first time point, an amount of expression of Axl, HPK1, or both to obtain a first expression level for Axl, HPK1, or both; (b) administering to the subject a therapeutically effective amount of an Axl inhibitor and an HPK1 inhibitor; (c) measuring in a sample from the subject, obtained at a subsequent time point, an amount of expression of Axl, HPK1, or both to obtain a second expression level of Axl, HPK1, or both; and (d) comparing the first expression level to the second expression level to determine whether a change in expression level has occurred. The method may further include, administering a therapeutically effective amount of an Axl inhibitor and an HPK1 inhibitor at a second time point subsequent to treatment at a first time point. In some embodiments, the combination therapy is used on a subject that has previously failed to respond to an immune checkpoint inhibitor. In some embodiments, the HPK1 inhibitor is HPK1-IN-7 or GNE-1858. In some embodiments, the Axl inhibitor is bemcentinib. In some embodiments, the methods of monitoring may include measuring in the sample at the first and/or second time point an amount of expression of Kras^{G12D}. In some embodiments, the method of monitoring may further include, in step (b), administering to the subject a therapeutically effective amount of a Kras^{G12D} inhibitor and/or a CD137 agonist antibody.

### V. Kits

The Axl inhibitor, HPK1 inhibitor, Kras^{G12D} inhibitor, and optionally CD137 agonist antibody combination therapy disclosed herein may be used for the preparation of a kit (e.g., a kit for the treatment of a patient). In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the method.

In one aspect, kits are provided for the determination of the likelihood of a subject with cancer responding to a therapy, wherein the kits can be used to first detect the biomarkers associated with said cancer. The kit may include, e.g., one or more agents for the detection of biomarkers, a container for holding a biological sample, e.g., blood sample or bone marrow sample, isolated from a human subject with cancer; and instructions for reacting agents with the biological sample or a portion of the biological sample to detect the presence or amount of at least one biomarker in the biological sample. The agents may be packaged in separate containers. The kit may further comprise one or more control reference samples and reagents for performing the herein-described methods. The kit may also comprise one or more devices or implements for carrying out any of the herein methods.

An Axl inhibitor, an HPK1 inhibitor, a Kras^{G12D} inhibitor, and a CD137 agonist antibody as described in this disclosure for use in treating cancer patients may be delivered in a pharmaceutical package or kit to doctors, healthcare providers, treatment facilities, or cancer patients. Such packaging is intended to improve patient convenience and compliance with the treatment plan. Typically, the packaging comprises paper (cardboard) or plastic. In some embodiments, the kit or pharmaceutical package further comprises instructions for use (e.g., for administering according to a method as described herein).

In some embodiments, a pharmaceutical package or kit comprises unit dose forms of an Axl inhibitor, an HPK1 inhibitor, a Kras^{G12D} inhibitor, and a CD137 agonist antibody. In some embodiments, the pharmaceutical package or kit further comprises unit dose forms of one or more of a chemotherapeutic agent, a cytotoxic agent, a radiotherapeutic agent, or an immunotherapeutic agent.

The kit can comprise one or more containers for compositions contained in the kit Compositions can be in liquid form or can be lyophilized. Suitable containers for the compositions include, for example, bottles, vials, syringes, and test tubes. Containers can be formed from a variety of materials, including glass or plastic.

In one embodiment, the kit or pharmaceutical package includes doses suitable for multiple days of administration, such as one week, one month, or three months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### EXAMPLES

The following examples are offered to illustrate, but not to limit, the claimed invention.

Ethics statement. All animal work performed was approved by MD Anderson's Institutional Animal Care and Use Committee. All animals were maintained in pathogen-free conditions and care for in accordance with the International Association for Assessment and Accreditation of Laboratory Animal Care policies and certification.

### Example 1. Methods and Materials

HPK1 knockout (HPK1^{-/-}) mice and transgenic mice expressing either HPK1 or kinase-dead HPK1 mutant, M46, were generated for studying the functions of HPK1 in the tumorigenesis and progression of PDAC, to test therapeutic interventions, and to study the critical role of the tumor microenvironment in pancreatic ductal adenocarcinoma (PDAC). Pancreas from HPK1^{-/-}/L/IPF (HPK1^{-/-}; LSL-Kras^{G12D}; Pdx-Cre) or L/IPF (LSL-Kras^{G12D};, Pdx-Cre) mice and spleen were isolated from HPK1^{-/-} or wild type control, and cell lysates were prepared from the corresponding sample. The cell lysates were separated by 10 % SDS-PAGE, then electroblotted onto polyvinylidene difluoride membranes (Novex, Grand Island, NY). The membranes were subsequently blocked in 5 % skim milk in 1x tris buffer saline with Tween 20 (TBST). Following blocking in 5 % milk, the samples were subsequently washed with PBS before incubating with primary antibodies for Axl and HSP90 as a control.

For flow cytometry studies, orthotopic xenograft tumors of KPC-Luc cells from the HPK1−/− or wild type control were digested in digestion buffer (1 mg/mL collagenase IV and 10 U/mL DNase I in Dulbecco's Modified Eagle Medium (DMEM)) at 37° C. for 30 minutes. After thorough mixing, the samples were pushed through a 70-μm cell strainer and washed with 40 ml DMEM without fetal bovine serum (FBS), The digested cells were re-suspended in 37 % Percoll and overlaid on top of the same volume of 70% Percoll. After centrifugation at 800 g for 20 minutes with break off, the lymphocytes were isolated from the interface and rinsed with PBS. The cells were first stained by Zombie UV, followed by incubation in a cocktail comprising the following antibodies in cell staining buffer: anti-CD45, anti-CD38, anti-B220, anti-CD11b, anti-F4/80, anti-Ly6C, anti-Ly6G, anti-CD4, anti-IA/IE, and anti-CD8g. After washing, the stained cells were submitted for multiple-color flow cytometry. The results were processed using FlowJo software.

For sub-cutaneous xenograft studies, the C57BL/6 female or male mice aged 5-7 weeks (Jackson Labs) were used. 1×10⁶ KPC-Luc cells or 2×10⁵ B16F10 melanoma cells were injected into the dorsal region of the wild type (WT) or HPK1-mice. Animals were imaged using the IVIS Spectrum, PerkinElmer and Bruker ICON MRI at 3 and 10 days after successful implantation of KPC-Luc cells. Only mice with similar tumor volume (~150 mm³) were used for treatment studies. These experiments were randomized. The animal cohort sizes were estimated based on previous experience using similar mouse models that showed significance. Mice were euthanized for tumor collection once tumor volume was approximately ~1000 mm³. For orthotopic studies, 0.5×10⁶ KPC-Luc cells were implanted orthotopically into WT C57BL/6 mice and/or HPK1^{-/-} mice pancreas. Bioluminescence imaging with the IVIS Spectrum (Perkin Elmer) was performed after intraperitoneal injection of 1.5 mg of D-luciferin (PerkinElmer). After image acquisition, the Living Image 4.7 software (PerkinElmer) was used for analysis of images. Therapy was initiated at day 10 post tumor cell injection. All mice were sacrificed on Day 35 for survival study or otherwise labeled in the Figures.

To examine the effects of pharmacological inhibition of Axl, Kras, and HPK1 with or without Anti-41BB/CD137 on tumor growth, Axl inhibitor (Bemcentinib MedChem Express) was dosed at 10 mg/kg/day for intraperitoneal injection (IP). HPK1-IN-7 (HPK1 inhibitor #1 MedChem Express) was dosed at 20 mg/kg/day orally, GNE1858 (HPK1 inhibitor #2 MedChem Express) was dosed at 10 mg/kg/day orally. KRAS^{G12D} inhibitor (MRTX1133, MedChem Express) was dosed at 25 mg/Kg twice daily for intraperitoneal injection. Anti-41BB (clone LOB12.3, BioXCell, BE0169) antibody and its corresponding isotype IgG controls were intraperitoneally administered at 200 μg per injection for three times per week. The duration of treatment was 10 days. The mice were sacrificed, and the tumors were weighed and harvested for Western blot as indicated in the figures. Tumor size was measured every other day using the methods as previously described.

For TCGA GSEA analysis, the TCGA PDAC mRNA dataset was downloaded from Broad GDAC website. All data processing and analysis were implemented in R 4.0.5 environment and Seurat package version 4.0.1.

RNA-seq was performed at MD Anderson ATGC with NovaSeq, generating 100-bp pair end reads. The sample library was prepared using Illumina TruSeq stranded protocol. RNA-seq FASTQ files were processed through FastQC, a quality control tool to evaluate the quality of sequencing reads at both the base and read levels. Samples passed QC were taken into subsequent analysis. STAR alignment (Dobin, et al., Bioinformatics., 29:15-21 (2013)) to the GRCm38 was performed with default parameters to generate RNA-seq BAM files. Aligned reads were summarized at the gene level using STAR (Dobin, et al., Bioinformatics., 29:15-21 (2013)). Gene-level annotation was carried out using the GENCODE annotation, which was downloaded from the GENCODE project (Harrow et al., Genome Res., 22(9):1760-74 (2012)). The raw count data was processed and normalized by Deseq2 (Love et al., Genome Biol., 15(12):550 (2014 )) software to identify differentially expressed genes (DEGs) between two groups. The final p-value was adjusted using the Benjamini and Hochberg method (Benjamini & Hochberg, J. Royal Stat. Soc. Series B-Stat. Meth., 57(1):289-300(1995)). A cut-off of gene expression log2 fold change of>=1.0 or<=−1.0 and an FDR q-value of<=0.05 was applied to select the most significant DEGs. Differential expression analysis was further evaluated utilizing pathway enrichment tool GSEA (Subramanian et al., Proc. Natl. Acad. Sci. USA., 102(43):15545-50 (2005)).

### Example 2. Expression Profile of HPK1 Knockout Mice

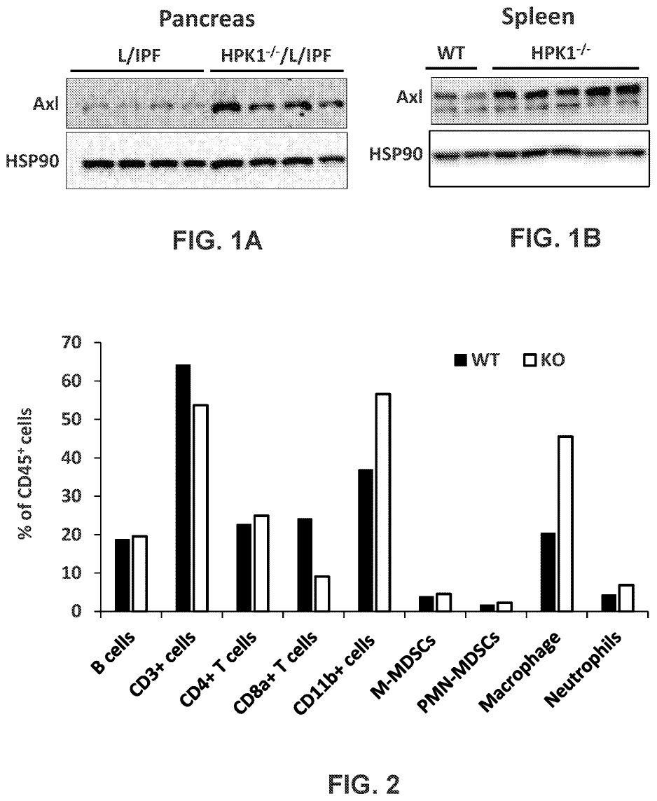

The HPK1 knockout mice have an increased expression of Axl both in the pancreas and spleen, which consists of predominantly lymphocytes, dendritic cells, and macrophages, when compared to the wild type control mice (FIG. 1).

To understand the cellular profile of HPK1 knockout mice, flow cytometry was performed on cell lysates prepared from the xenograft tumor from both the wild type and HPK1 knockout mice. It was determined that the knockout HPK1 mice have increased intra-tumoral macrophages and myeloid-derived suppressor cells (CD11b+ cells) and reduced CD8+ T cells. Not bound by any theory, it is hypothesized the reduced number of T cells is due to the upregulation of Axl in the immune cells.

### Example 3. Treatment with an Axl Inhibitor Decreased Tumor Growth in HPK1 Knockout Mice

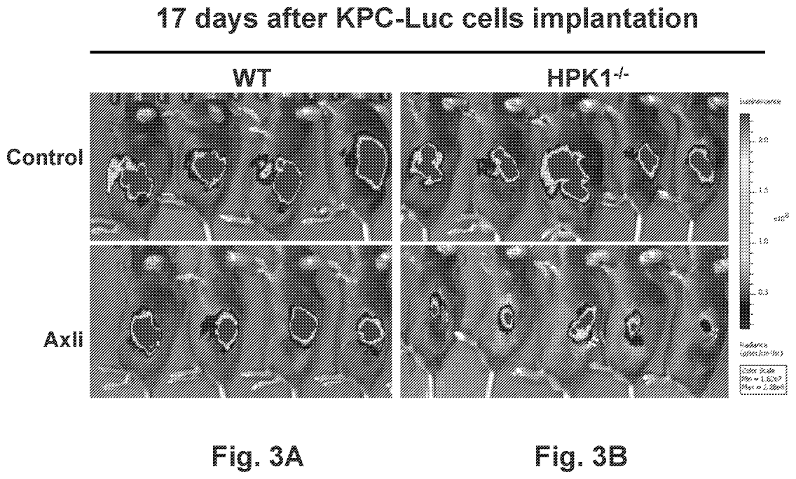

To evaluate the effects an Axl inhibitor has on the cellular profile of both wild type and HPK1 knockout mice, orthotopic xenograft tumors of pancreatic cancer in HPK1 knockout mice and wild type mice were imaged after 17 days post implantation with KPC-Luc cells. The wild-type mice demonstrated a larger tumor growth as shown by the increased luminescence (FIG. 3A). Treatment of the wild type mice with the Axl inhibitor (bemcentinib) resulted in little to no decrease in the tumor volume. The HPK1 mice left untreated showed a larger tumor volume, while treatment with the Axl inhibitor (bemcentinib) inhibited the growth of the tumor (FIG. 3B).

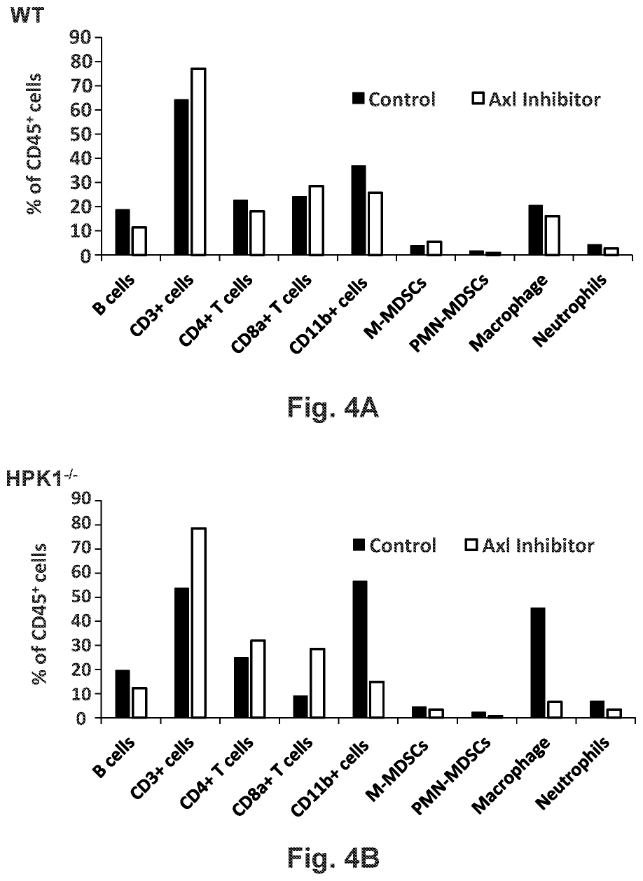

Following tumor xenograft imaging, the tumors were excised from the mice and subsequently prepared for flow cytometry as described above. The introduction of the Axl inhibitor in wild-type mice did not increase or decrease the number or function of B cells, CD3+ cells, CD4+ T cells, CD8+ T cells, CD11b+ cells, M-MDSC's, PMN-MDSC's, Macrophages, or neutrophils (FIG. 4A).

Interestingly, introducing Axl inhibitor bemcentinib (R248) into the HPK1 knockout mice demonstrated a synergistic type of response. The Axl inhibitor decreased the infiltration of macrophages and myeloid suppressor cells and increased the CD3+ T cells and CD8+ T cells in the tumor microenvironment of the pancreatic cancer (FIG. 4B).

### Example 4. Axl Expression Correlates with Cytokines and Markers for Macrophages and Myeloid Suppressor Cells in Mouse Pancreas and Human Pancreatic Cancer Samples

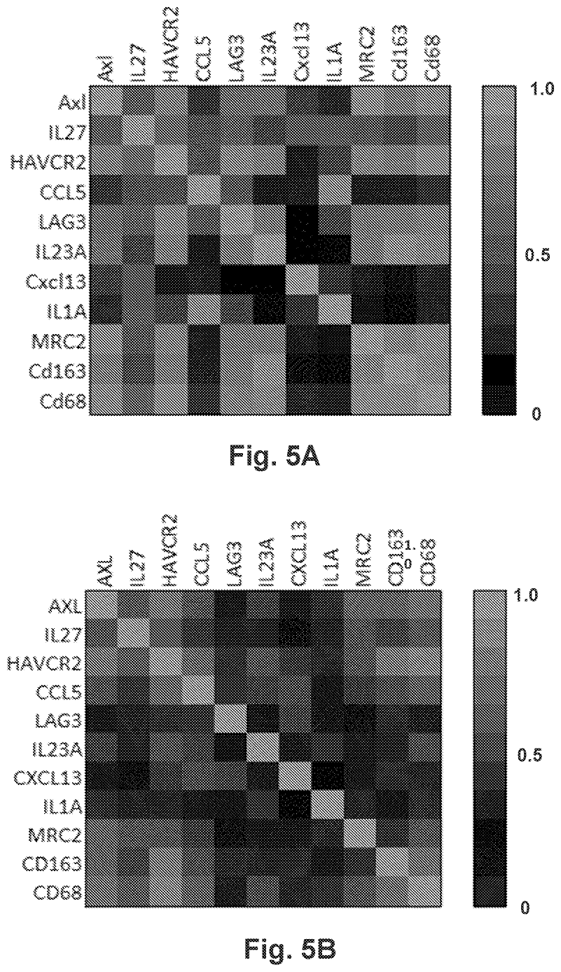

RNA sequencing analysis was performed as described above to understand the correlation of Axl expression with the expression of cytokines in both human pancreatic cancer samples and mouse pancreas. The RNA sequencing analysis show that Axl expression correlated with the upregulation of multiple immunosuppressive cytokines or markers for macrophages and myeloid suppressor cells in the mouse pancreas (FIG. 5A). RNA sequencing analysis of cells in human pancreatic cancer samples based on TCGA data sets revealed that Axl expression correlates with the upregulation of multiple immunosuppressive cytokines or markers for macrophages and myeloid suppressor cells (FIG. 5B).

Knockout HPK1 markedly decreases the expression of 4-1BB (CD 137) in mouse pancreas (data not shown). CD137 promotes the survival of activated T cells, suggesting that HPK1 inhibitor with a CD137 agonist may enhance the effect of HPK1 in cancer immunity and immune-mediated killing of tumor cells.

### Example 5. Combination Treatment with an Axl Inhibitor and HPK1 Inhibitor Significantly Increases Anti-Tumor Activity.

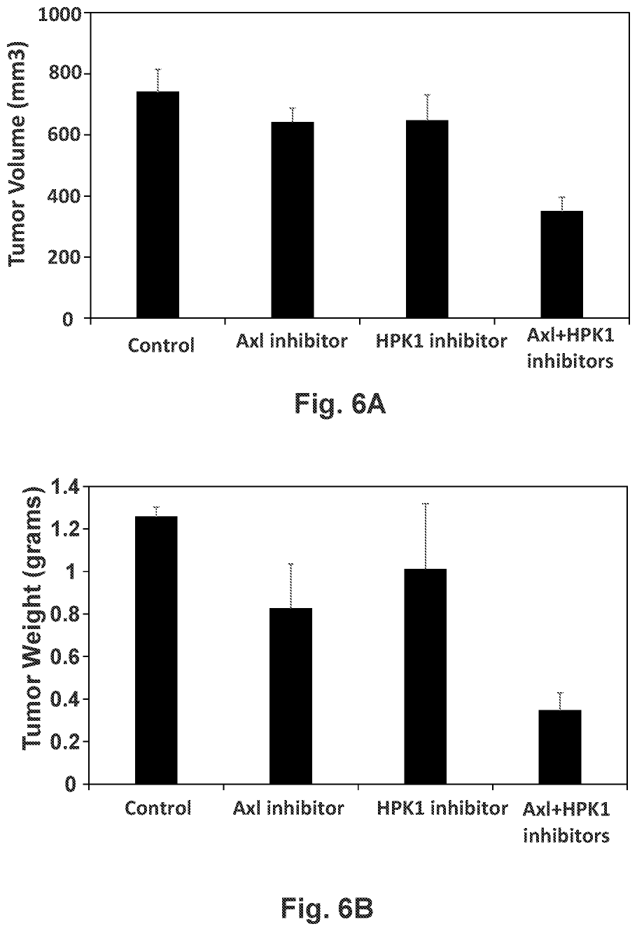

Tumors were grown in wild-type mice, and treatment was performed as described above. Following treatment, tumors were excised from the mice and both tumor volume and tumor weight were measured. Combined treatment of Axl inhibitor, bemcentinib, with an HPK1 inhibitor (HPK1-IN-7) significantly improved the anti-tumor efficacy compared to the groups treated with either HPK1 inhibitor or Axl inhibitor alone (FIGS. 6A and 6B). The tumor volume did not significantly decrease upon treatment with Axl inhibitor or HPK1 inhibitor alone, while treatment with a combination of HPK1 and Axl inhibitor significantly reduced the tumor volume (FIG. 6A). A similar trend was observed in the tumor weight as with the tumor volume. In particular, the Axl inhibitor alone, reduced the tumor weight to a small degree while the HPK1 inhibitor alone did not reduce the tumor weight when compared to the control. However, the combination therapy of the Axl inhibitor with the HPK1 inhibitor the tumor weight was significantly decreased when compared to the control group (FIG. 6B).

### Example 6. Combination Treatment of an Axl Inhibitor, HPK1 Inhibitor, and CD137 Agonist Antibody Significantly Increases Anti-Tumor Activity

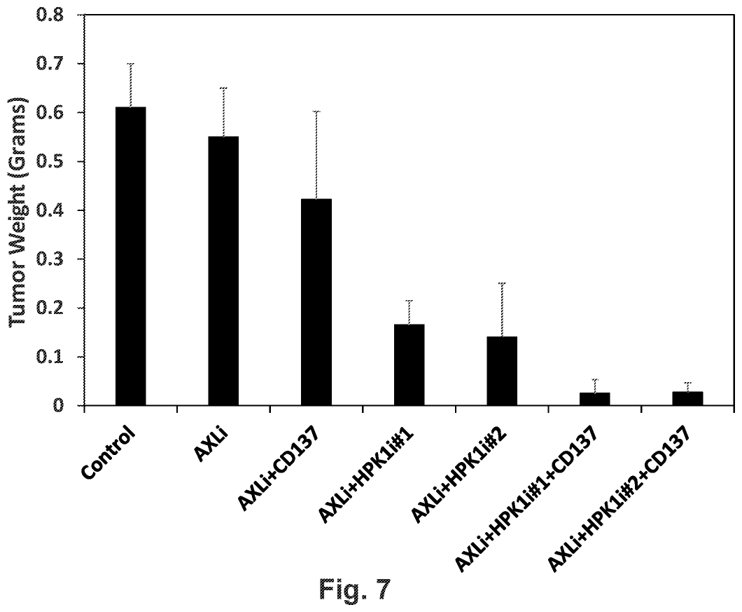

To further study the synergistic effects of the combination therapy, tumors were grown in wild-type mice and treatment was performed as described above. Following treatment, tumors were excised from the mice and tumor weight was evaluated. The control tumor weight was about 0.6 grams while treatment with Axl inhibitor only minimally reduced the tumor volume. Treatment with a combination therapy of Axl and CD137 additionally did not reduce the tumor weight when compared to the control. Treatment with Axl inhibitor and HPK1 inhibitors (HPK1-IN-7 (#1) or GNE-1858 (#2)) reduced the tumor volume to about 0.2 grams (P<0.001). Interestingly, the tumor weight from mice treated with a triple combination therapy (Axl inhibitor, HPK1 inhibitor and CD137 agonist antibody) demonstrated a significant reduction in tumor weight to below 0.1 grams (P<0.0001)(FIG. 7). The results demonstrate a surprising and unexpected synergistic effect of the Axl inhibitor and HPK1 inhibitor alone or further in combination with a CD137 agonist antibody for the treatment of PDAC.

### Example 7. Treatment with a KRASGED Inhibitor Decreased Tumor Growth in HPK1 Knockout Mice

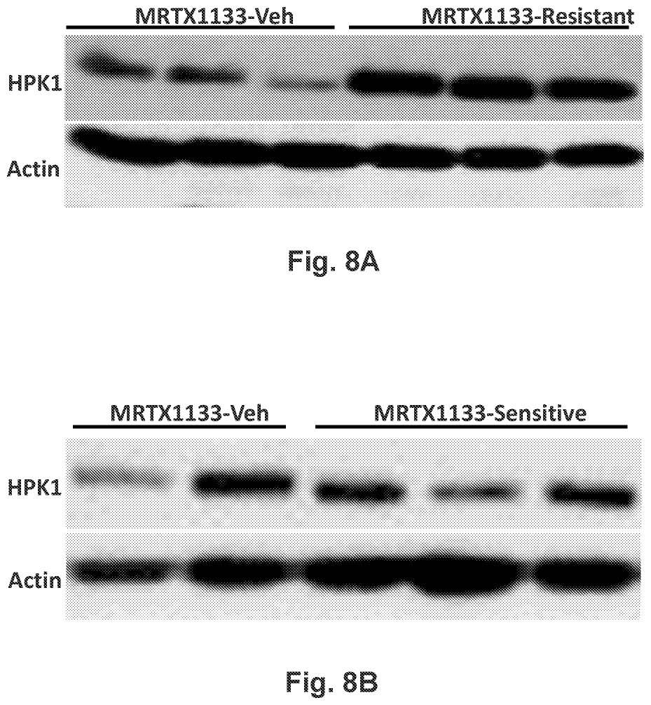

To analyze the association of HPK1 with resistance to KRAS^{G12D} inhibitors, expression of HPK1 was determined by Western blot analysis of splenic lymphocytes isolated from the spleen of C57BL/6 mice with orthotopic KPC-Luc tumors having developed resistance to the KRAS^{G12D} inhibitor MRTX1133 (FIG. 8A) or tumors sensitive to MRTX1133 (FIG. 8B). The results demonstrate that HPK1 is upregulated in MRTX1133-resistant mice. This upregulation may be associated with tumor resistance to MRTX1133.

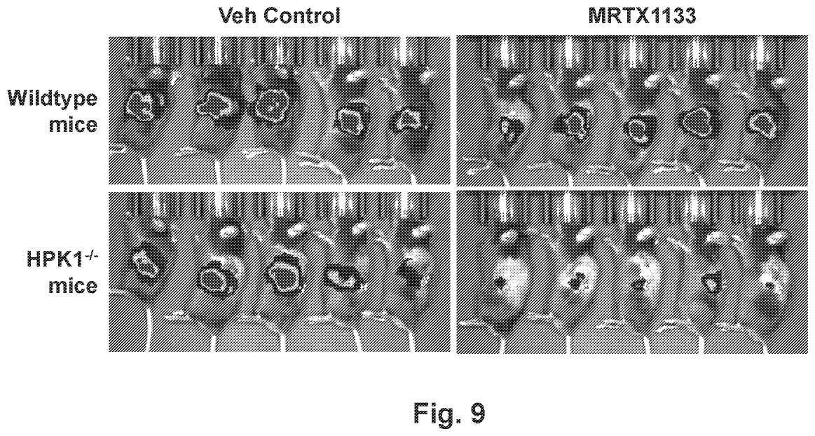

To further evaluate the association, tumor size was determined in mice treated with MRTX1133. Orthotopic xenograft tumors of pancreatic cancer were introduced in HPK1 knockout mice and wild type mice by implantation with 1×10⁶ KPC-Luc cells. After 10 days, the mice were treated with MRTX1133 (25 mg/kg, intraperitoneal injection, twice daily) or a vehicle control, and these tumors were then imaged 7 days after treatment (FIG. 9). The wild-type mice treated with MRTX1133 (top right panel) were less responsive than the HPK1 knockout mice to treatment with MRTX1133 (bottom right panel), as demonstrated by tumor size shown by luminescence.

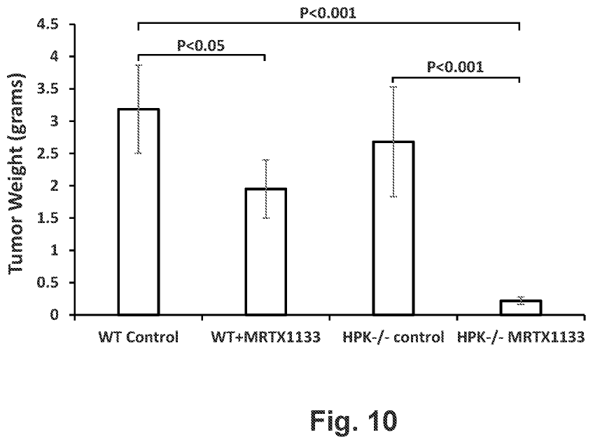

Further demonstrating the therapeutic effect of MRTX1133 on HPK1 knockout mice, tumor weight (grams) was evaluated in mice tumor models (FIG. 10). Specifically, wild type and HPK1 knockout mice having orthotopic KPC-Luc pancreatic tumors were treated with 25 mg/kg of MRTX1133 twice daily for ten days. The tumors were subsequently harvested and weighed seven days after the completion of treatment with MRTX1133. Wild type mice demonstrated a slight decrease in tumor weight while HPK1 knockout mice treated with MRTX1133 demonstrated a significant decrease in tumor weight.

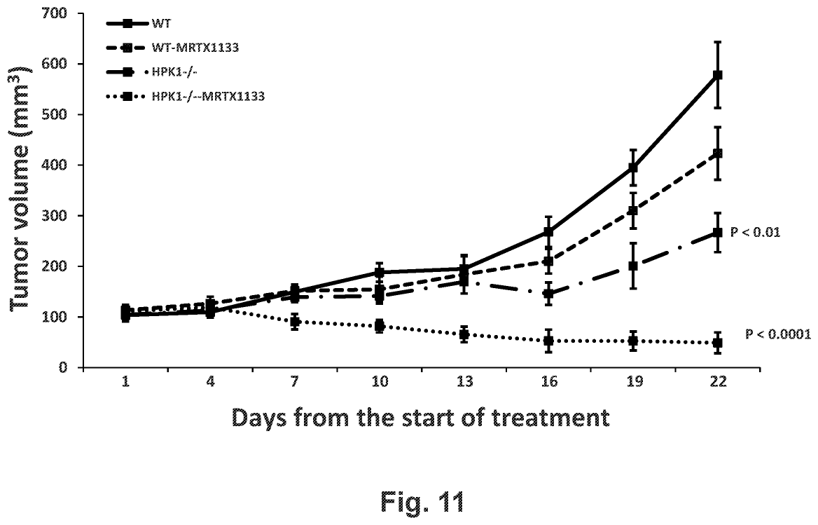

To further evaluate the therapeutic effect MRTX1133 has on the cellular profile of both wild type and HPK1 knockout mice, subcutaneous tumors of pancreatic cancer (1×10⁶ KPC-Luc) in HPK1 knockout mice and wild type mice were measured for tumor volume (mm³) (FIG. 11). Specifically, wild type and HPK1 knockout mice were subcutaneously injected with KPC-Luc pancreatic cancer cells. Following 10 days of growth, wild type and HPK1 knockout mice were treated with 10 mg/kg of MRTX1133 twice a day for ten days. Tumor volume was assessed continually from the start of treatment to 12 days post treatment. Taken collectively, the results demonstrate the potential therapeutic effect of combination therapies using HPKi and Axli inhibitors to overcome resistance in PDAC xenograft models. The results demonstrated the potential ability for the methods described herein to be used in other cancer types presenting with Ras mutations.

### Example 8. Treatment with an Axl Inhibitor Decreased Tumor Growth of Xenograft Melanoma Model in HPK1 Knockout Mice

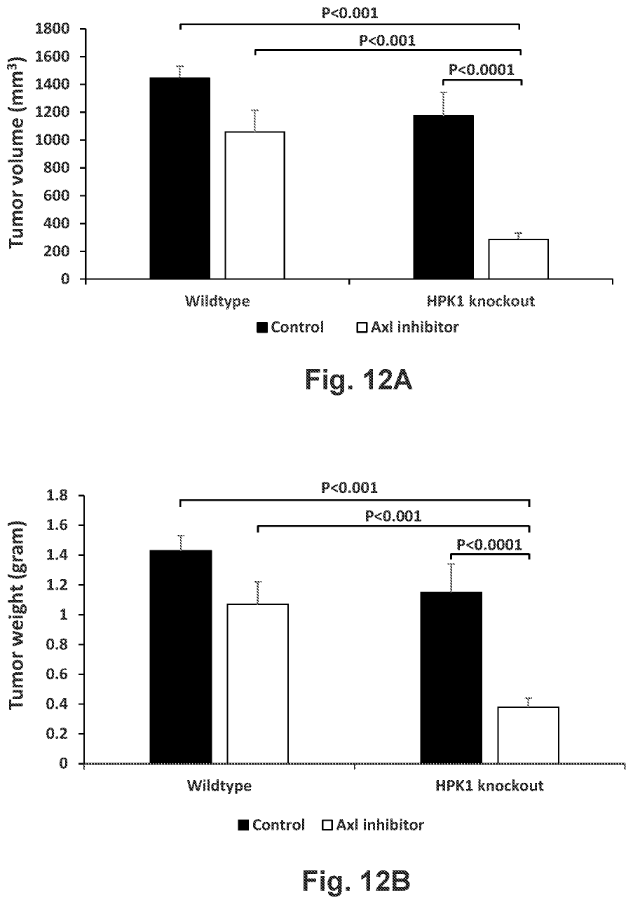

To evaluate the therapeutic effect of an Axl inhibitor, bemcentinib, on melanoma, subcutaneous tumors of subcutaneous melanoma xenograft model of B16F10 cells in HPK1 knockout mice and wild type mice were treated with 10 mg/kg/day bemcentinib by intraperitoneal injection (IP) twice daily for 10 days. The tumor volumes (FIG. 12A) and tumor weight (FIG. 12B) were measured at 12 days after the completion of treatment. Wild type mice demonstrated a slight decrease in tumor volume and weight, while HPK1 knockout mice treated with bemcentinib demonstrated a significant decrease in tumor volume and weight.

### Example 9. Combination Treatment with an Axl Inhibitor and HPK1 Inhibitor Significantly Increases Anti-Tumor Activity on Melanoma

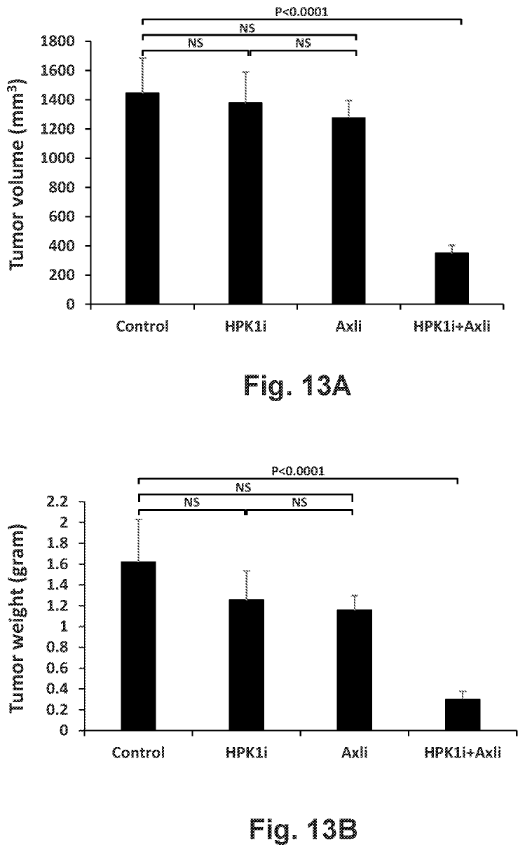

Subcutaneous melanoma xenograft tumors of B16F10 cells were grown in wild-type mice, and treatment was performed as described above. Following treatment, tumors were excised from the mice at day 12 after completion of therapy and both tumor volume and tumor weight were measured. Combined treatment of Axl inhibitor, bemcentinib, with an HPK1 inhibitor (HPK1-IN-7) significantly improved the anti-tumor efficacy compared to the groups treated with either HPK1 inhibitor or Axl inhibitor alone (FIGS. 13A and 13B). The tumor volume did not significantly decrease upon treatment with Axl inhibitor or HPK1 inhibitor alone, while treatment with a combination of HPK1 and Axl inhibitor significantly reduced the tumor volume (FIG. 13A). A similar trend was observed in the tumor weight as with the tumor volume. In particular, either Axl inhibitor or HPK1 inhibitor alone, reduced the tumor weight to a small degree when compared to the control. However, the combination therapy of the Axl inhibitor with the HPK1 inhibitor significantly decreased the tumor weight when compared to the control group (FIG. 13B).

Taken collectively, the results demonstrate the potential therapeutic effect of combination therapies using HPK1 and Axl inhibitors on melanoma in melanoma xenograft models.

The above description of example embodiments of the present disclosure has been presented for the purposes of illustration and description. It is not intended to be exhaustive or to limit the disclosure to the precise form described, and many modifications and variations are possible in light of the teaching above.

1. A method of treating a subject that has or is suspected of having cancer, comprising administering to the subject a therapeutically effective amount of an Axl inhibitor and an HPK1 inhibitor.

2. (canceled)

3. The method of claim 1, wherein the HPK1 inhibitor is selected from a group consisting of HPK1-IN-7, GNE-1858, GNE-6893, BGB-15025, CFI-402411, and compound K (CompK).

4. (canceled)

5. The method of claim 1, wherein the Axl inhibitor is selected from a group consisting of bemcentinib, SLC-391, DS-1205c, gilteritinib, BPI-9016M, INCB081776, PF-07265807, Q702, TP-0903, and monoclonal antibodies YW327.6S2, D9, and E8.

6. The method of claim 1, wherein the HPK1 inhibitor is HPK1-IN-7 or GNE-1858, and wherein the Axl inhibitor is bemcentinib.

7. The method of claim 6, further comprising administering to the subject a therapeutically effective amount of a Kras^{G12D} inhibitor, wherein the Kras^{G12D} inhibitor is MRTX1133.

8. (canceled)

9. The method of claim 1, further comprising administering to the subject a therapeutically effective amount of a CD137 agonist antibody.

10. The method of claim 1, wherein the cancer is selected from a group consisting of pancreatic, skin, lung, colon, prostate, breast, ovary, esophageal, stomach, or kidney.

11.-13. (canceled)

14. The method of claim 1, further comprising at least a second anti-cancer therapy.

15. The method of claim 14, wherein the second anti-cancer therapy is a chemotherapy, molecular targeted therapy, immunotherapy, gene therapy, surgery, hormonal therapy, epigenetic modulation, anti-angiogenic therapy, or cytokine therapy.

16. A method of monitoring a response of a subject to a combination therapy, comprising:
(a) measuring in a sample from a subject, obtained at a first time point, an amount of expression of Axl, HPK1, or both to obtain a first expression level for Axl, HPK1, or both;
(b) administering to the subject a therapeutically effective amount of an Axl inhibitor and an HPK1 inhibitor;
(c) measuring in a sample from the subject, obtained at a subsequent time point, an amount of expression of Axl, HPK1, or both to obtain a second expression level of Axl, HPK1, or both; and
(d) comparing the first expression level to the second expression level to determine whether a change in expression level of Axl, HPK1, or both has occurred.

17. The method of claim 16, wherein the HPK1 inhibitor is HPK1-IN-7 or GNE-1858, and wherein the Axl inhibitor is bemcentinib.

18. (canceled)

19. The method of claim 16, further comprising:
(e) measuring in the sample from the subject, obtained at the first time point, an amount of expression of Kras^{G12D} to obtain a first Kras^{G12D} expression level;
(f) optionally administering to the subject a therapeutically effective amount of a Kras^{G12D} inhibitor;
(g) measuring in the sample from the subject, obtained at the subsequent time point, an amount of expression of Kras^{G12D} to obtain a second expression Kras^{G12D} level; and
(h) comparing the first Kras^{G12D} expression level to the second Kras^{G12D} expression level to determine whether a change in Kras^{G12D} expression level has occurred.

20. The method of claim 16, further comprising administering to the subject a therapeutically effective amount of a CD137 agonist antibody before the subsequent time point.

21. The method of claim 16, wherein the subject has or is suspected of having a cancer, wherein the cancer is selected from a group consisting of pancreatic, skin, lung, colon, prostate, breast, ovary, endometrial, liver, esophageal, stomach, or kidney.

22.-24. (canceled)

25. The method of claim 16, further comprising administering to the subject at least a second anti-cancer therapy before the subsequent time point, wherein the second anti-cancer therapy is a chemotherapy, molecular targeted therapy, immunotherapy, gene therapy, surgery, hormonal therapy, epigenetic modulation, anti-angiogenic therapy, or cytokine therapy.

26.-28. (canceled)

29. A method of determining a likelihood that a subject will respond to an Axl-HPK1 inhibitor combination therapy, the method comprising:
(a) obtaining a sample from a subject;
(b) detecting an expression level of HPK1 or Axl in the sample; and
(c) indicating that the subject is likely to respond to treatment if the subject's cancer is characterized as having high levels of HPK1 or Axl expression.

30. The method of claim 29, further comprising administering to the subject an HPK1 inhibitor and an Axl inhibitor if the expression level detected in step (b) is above a threshold level.

31. The method of claim 29, wherein the expression level is a protein expression level.

32. The method of claim 29, wherein the expression level is a nucleic acid expression level.

33. (canceled)

34. The method of claim 1, wherein the Axl inhibitor and the HPK1 inhibitor modulate macrophages, CD8+ T cells, CD3+ T cells, CD4+ T cells, myeloid suppressor cells, or any combination thereof.

35.-45. (canceled)
